(19) United States
(12) Patent Application Publication
(10) Pub. No.: US 2009/0105962 A1
(43) Pub. Date: Apr. 23, 2009

(54) METHODS AND SYSTEMS FOR IDENTIFYING MOLECULAR PATHWAY ELEMENTS

(75) Inventors: Peter Woolf, Ann Arbor, MI (US); Yongqun He, Ann Arbor, MI (US); Andrew Hodges, Ann Arbor, MI (US)

Correspondence Address: Casimir Jones, S.C., 440 Science Drive, Suite 203, Madison, WI, 53711, US

(73) Assignee: The Regents of the University of Michigan, Ann Arbor, MI (US)

(21) Appl. No.: 12/139,529

(22) Filed: Jun. 16, 2008

Related U.S. Application Data
(60) Provisional application No. 60/934,581, filed on Jun. 14, 2007.

(51) Int. Cl. G01N 33/48

(52) U.S. Cl. 702/20

(57) ABSTRACT

The present invention provides methods and systems useful in determining molecular pathways and elements of molecular pathways. In particular, the present invention provides for the discovery of new molecular pathway elements using Bayesian networks and gene expression information.

This application claims priority to U.S. Provisional Application No. 60/934,581, filed Jun. 14, 2007, herein incorporated by reference in its entirety.

This invention was made with government support under Grant Numbers U54-DA-021519, AI057875 and HG003890 awarded by the National Institute of Health. The government has certain rights in the invention.

### FIELD OF THE INVENTION

The present invention provides methods and systems useful in determining molecular pathways and elements of molecular pathways. In particular, the present invention provides for the discovery of new molecular pathway elements using Bayesian networks and gene expression information.

### BACKGROUND OF THE INVENTION

Pathway models and microarray gene expression data are often the first resources used when searching for biological mechanisms. Two common questions asked of these resources are: (1) can gene expression data be used to successfully reconstruct a known pathway, and (2) what other molecular players should be included to more fully understand the mechanism. The difficulty in integrating these data is that gene expression and pathway models describe cellular behaviors at different levels. For example, gene expression data describes mRNA concentrations in a particular cell or tissue type, while pathway models reveal a partial picture of the protein biochemistry that is generally true for many cell types. Furthermore, gene expression data are often static, while pathway models are dynamic. However, in spite of these differences there is a tendency to assume that connections in a pathway model must also be reflected at the gene expression level. Merging gene expression data and pathway information incorrectly can yield confusing results.

As such, what are needed are methods and systems for identifying molecular pathways and pathway elements using gene expression analysis.

### SUMMARY OF THE INVENTION

The present invention provides methods and systems useful in determining molecular pathways and elements of molecular pathways. In particular, the present invention provides for the discovery of new molecular pathway elements using Bayesian networks and gene expression information.

Certain illustrative embodiments of the invention are described below. The present invention is not limited to these embodiments.

In some embodiments, the present invention provides methods for determining new molecular elements in a molecular pathway comprising providing gene expression data, a molecular pathway, and a BN+1 algorithm, and applying the gene expression data and the molecular pathway information to the algorithm thereby determining new molecular elements in said molecular pathway. In some embodiments, the gene expression data is derived from a microarray platform. In some embodiments, the molecular pathway is a known molecular pathway, for example as found in KEGG or other databases. In some embodiments, the BN+1 algorithm is a component of a computer software integrated system, such as the Molecular Annotation Resource for Integrating Microarrays with Bayesian Analysis, or MARIMBA, or another software system capable of performing the same functions and integrated analysis as that of MARIMBA. In some embodiments, the present invention determines new molecular elements in molecular pathways by generation a Bayesian network for the BN+1 algorithm. In some embodiments, the results generated from the BN+1 algorithm are displayed in, for example, two and/or three-dimensional plots, or cascade types of pictorials.

In some embodiments, the molecular pathway information is displayed, for example, as a two and/or three dimensional plot, a cascade flowchart, or other two or three pictorial representation of the molecular pathways and elements thereof, on a display device. Display devices include, but are not limited to, computer monitors (e.g., via the INTERNET or INTRANET), television screens, hand-held devices, and the like.

### DESCRIPTION OF THE FIGURES

FIG. 1 shows the B-cell receptor (BCR) pathway in KEGG and corresponding genes in Bayesian network (BN) analysis.

FIG. 2 demonstrates the combined top Bayesian network of the B-cell receptor pathway. Eleven top-scoring BN networks were identified during the combined 72 day search for 42 gene BCR model, yielding the combined network. Inset demonstrates the marginalized probabilities for 30 of the 229 scored network classes for rank-ordered top BN models.

FIG. 3 shows the top 25 consensus interactions from BN analysis overlain on KEGG BCR pathway. The top 25 interactions were found from 3,281 networks (top 99.99% of marginalized probabilities).

FIG. 4 shows the top BN+1 network containing Rexo2. The gene Rexo2 regulated the expression of 15 other genes. Five interactions were changed after introduction of Rexo2 into the BCR pathway network. Inset demonstrates the marginized probability distribution for rank-ordered genes, showing the top-scoring network is significantly better than the other top networks found.

FIG. 5 shows exemplary two and three-dimensional plots of Rexo2 related relationships found by BN+1. (A) shows a two dimensional plot of Fos (child), Ikbkb and Rexo2 (parents). (B) shows a three dimensional plot of Rexo2 (parent) and B1nk (child).

FIG. 6 shows a conserved motif among all the 14 genes directly influences by Rexo2.

FIG. 7 shows a schematic workflow of the MARIMBA BN and BN+1 modeling pipelines.

FIG. 8 shows an executable flowchart for MARIMBA.

### DEFINITIONS

The term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and, where the RNA encodes a protein, into protein, through “translation” of mRNA. A “gene expression array” refers to the expression analysis of typically a large number of genes at one time. For example, Affymetrix® provides GeneChip® gene expression arrays wherein hundreds of genes at a time are queried for expression analysis.

The term “Bayesian network” refers to a probabilistic graphical model that represents a set of variables and their probabilistic dependencies. For example, a Bayesian network can be used to calculate the probability of a patient having a specific disease, given the absence or presence of certain symptoms, if the probabilistic dependencies between symptoms and disease are assumed to be known. In embodiments of the present application, a Bayesian network (BN and BN+1) algorithm has been developed (BN+1) to determine previously unknown molecular pathway elements of a given molecular pathway using a known pathway (e.g., as found in KEGG) and experimental gene expression analysis data.

### DETAILED DESCRIPTION OF THE INVENTION

Gene expression data and characterized biochemical pathways are both important for providing a mechanistic description of biology, but the two data types are often difficult to integrate. A new software program MARIMBA was developed for this integration, allowing both the comparison of pathway models and expression networks, and the rational addition of “hidden” genes that influence a pathway based on expression data. MARIMBA uses a Bayesian approach to merge these two data types in a meaningful way. Bayesian networks (BNs) are graphical models that describe causal or apparently causal interactions between variables. Bayesian networks are ideal for merging pathway models and gene expression data due to the network's flexibility, robustness to error, and human interpretability. Compared to other modeling methods, Bayesian networks are learned automatically by searching through the space of network topologies and retaining the most significant top-scoring networks. In systems biology, Bayesian networks have been used to identify relationships from gene expression data, protein interactions, and phosphorylation state regulation (Chen, 2007; Imoto, 2007; Woolf, 2005; Sachs, 2005; Deng, 2006). Bayesian networks have also been used for computational modeling of in silico genetic regulatory systems by using microarray gene expression data (Smolen, 2000). Other methods for network reconstruction, including statistical correlation (Petrie, 2000; Zou, 2003), neural networks (William, 1990), differential equation modeling (Perelson, 1993; Lauffenburger, 2003), and mutual information networks (Basso, 2005) are also powerful methods for this kind of analysis. However the rational incorporation of relational knowledge such as a pathway model is difficult using these non-Bayesian tools, and the results can be hard to interpret mechanistically.

In developing embodiments of the present invention, a pathway model and gene expression data were merged using methods and systems as described herein. As an example, pathway elements and computations were performed using the B-cell receptor pathway (BCR) as described by the Kyoto Encyclopedia of Genes and Genomes or KEGG (Kanehisa, 2004, Nucl. Acids. Res. 28:27-30) and gene expression data from perturbed B-cells obtained from the Alliance for Cellular Signaling (AfCS). The AfCS study gathered 629 microarray chips measuring gene expression in B cells from M musculus splenic extracts that are exposed to 32 different ligands (Lee, 2006; Zhu, 2004; Papin, 2004). This dataset is attractive because the same tissues were treated with combinations of ligands that perturb the B cell receptor signaling pathway.

The BCR pathway is part of the adaptive immune response mechanism by which lymphocytes respond to foreign antigens. The B cell receptor (BCR) pathway is involved in the activation of specific protein kinase C (PKC) isoforms that induces ultimate activation of the NF-κB transcription factor. NF-κB plays a crucial role in the antigen-induced B lymphocyte proliferation, cytokine production, and B cell survival (Lucas, 2004). The BCR pathway forms the cornerstone of the adaptive B cell immune response (Lucas, 2004). While the KEGG pathway database includes a manually curated BCR pathway, this pathway is still incomplete (Lucas, 2004). As such, systems and methods of the present invention were used to augment the BCR pathway based with the data provided by the AfCS dataset as an example of how systems and methods of the invention may be employed.

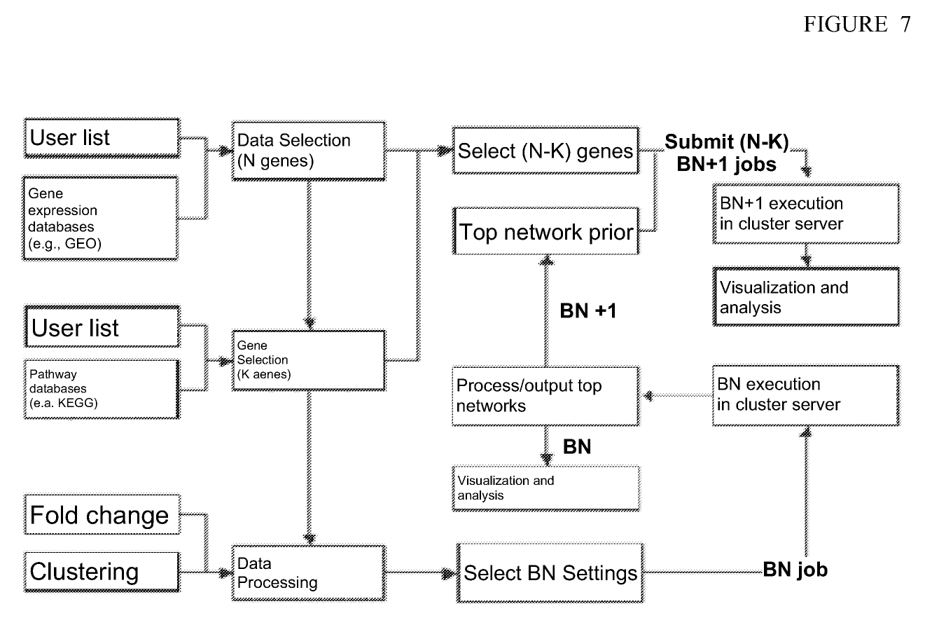

The analysis pipeline (FIG. 7) was implemented using a web-based system dubbed MARIMBA, available online at http://helab.bioinformatics.med.umich.edu/marimba. MARIMBA was used to reconstruct a regulatory model of the genes involved in the KEGG BCR pathway, and to identify additional genes outside the KEGG BCR pathway that are most influential in the BCR signaling pathway regulation using the novel “BN+1” algorithm as described herein.

### Comparison of Bayesian Network BCR Pathway to KEGG BCR Pathway

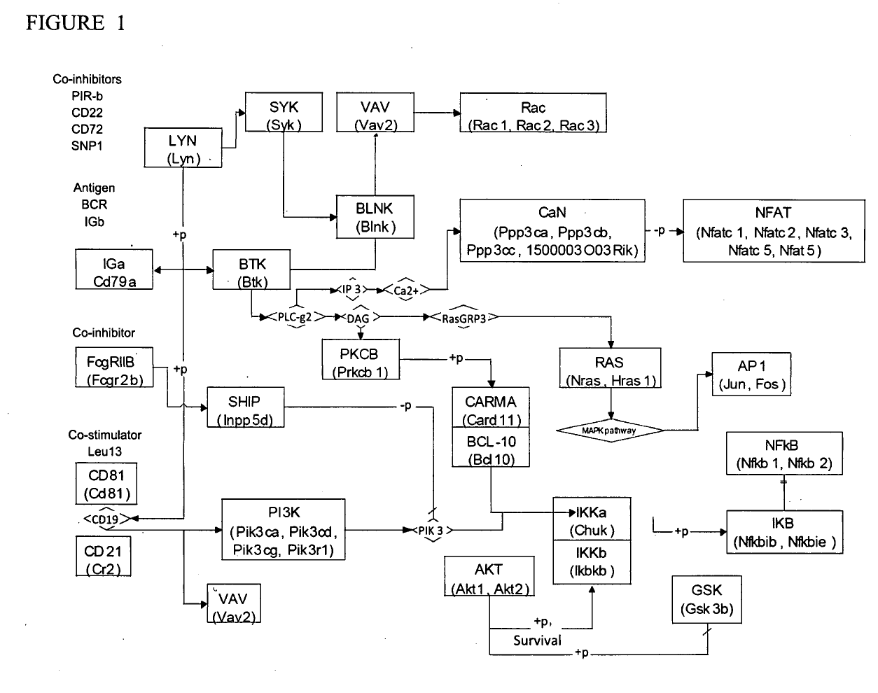

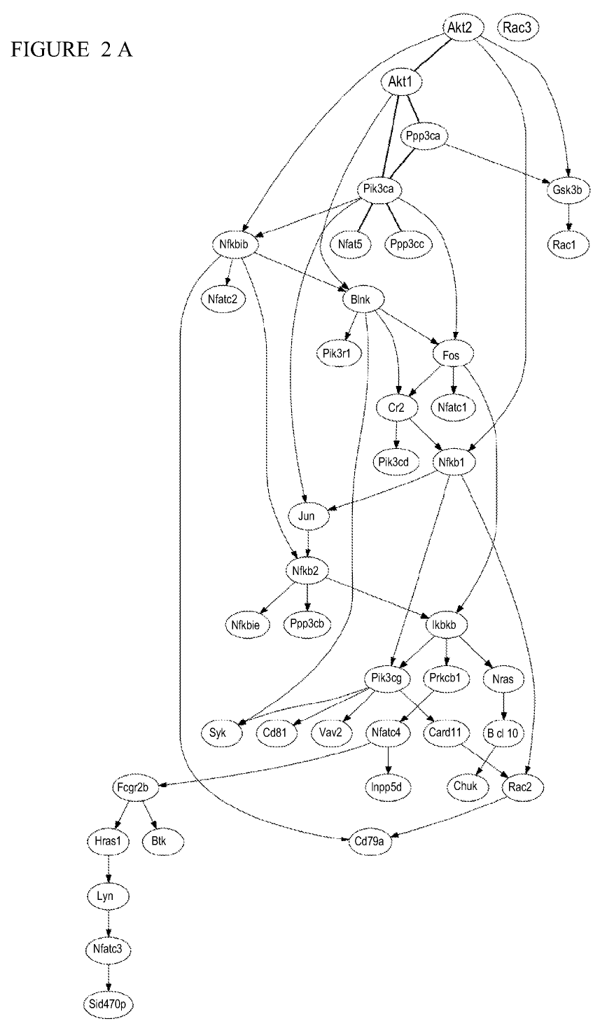

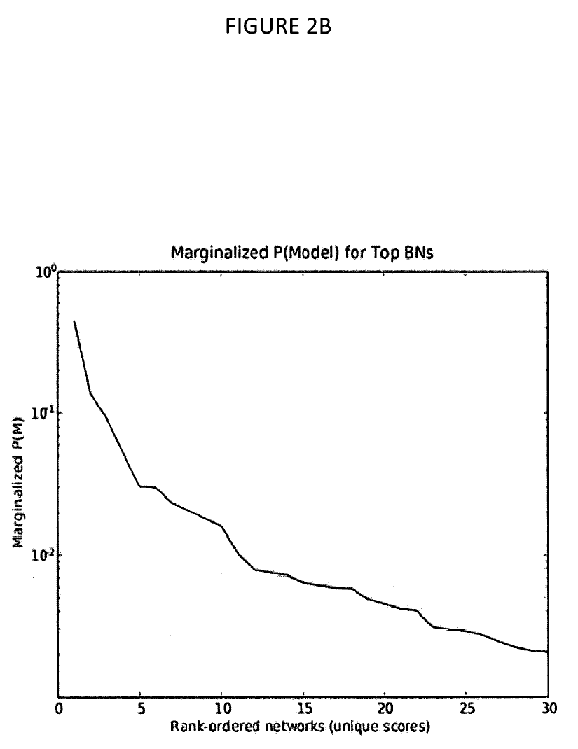

As an exemplary use of the present invention, the MARIMBA pipeline was used to create Bayesian networks from the AfCS expression data on 42 genes from the KEGG BCR pathway (KEGG ‘mmu04662’). The locations and gene-to-protein matching of these 42 genes in the KEGG BCR pathway were identified (FIG. 1). The core network was then identified from 1.55×10¹¹ networks learned over approximately 2000 hours of computing to generate a list of high-scoring BN networks for the 42 genes in the BCR pathway. Topping this list were 11 identically scoring networks, differing only in the directions of six conserved edges (FIG. 2). It was demonstrated that the top-scoring network was significantly better than the other top networks found, indicating that the top model captured the behavior well (FIG. 2). Examination of the second and third networks in the top-scoring list showed a near identical structure to the top network, differing by only a few connections.

The top-scoring network and KEGG network were compared. The comparison was classified into three relevant groups (Table 1).

TABLE 1
Comparison between KEGG BCR pathway and BN results

| | | |
|---|---|---|
| Gene 1 | Gene 2 | KEGG |

Group 1: identical relationship

| | | |
|---|---|---|
| Chuk | Bcl10 | |
| Gsk3b | Akt2 | +p, inhibition |
| Nfkbib | Nfkb2 | dissociation |
| Nfkb2 | Nfkbie | dissociation |

Group 2: same topology and inverted direction

| | |
|---|---|
| Blnk | Syk |
| Cd81 | Pik3cg |
| Pik3cg | Ikbkb |

Group 3: same neighborhood or Markov blanket

| | | |
|---|---|---|
| 1500003O0Rik | Nfatc3 | −p |
| Akt1 | Pik3ca | |
| Akt2 | Pik3ca | |
| Card11 | Pik3cg | |
| Fcgr2b | Inpp5d | |
| Ppp3cc | Nfat5 | |
| Vav2 | Cd81 | |

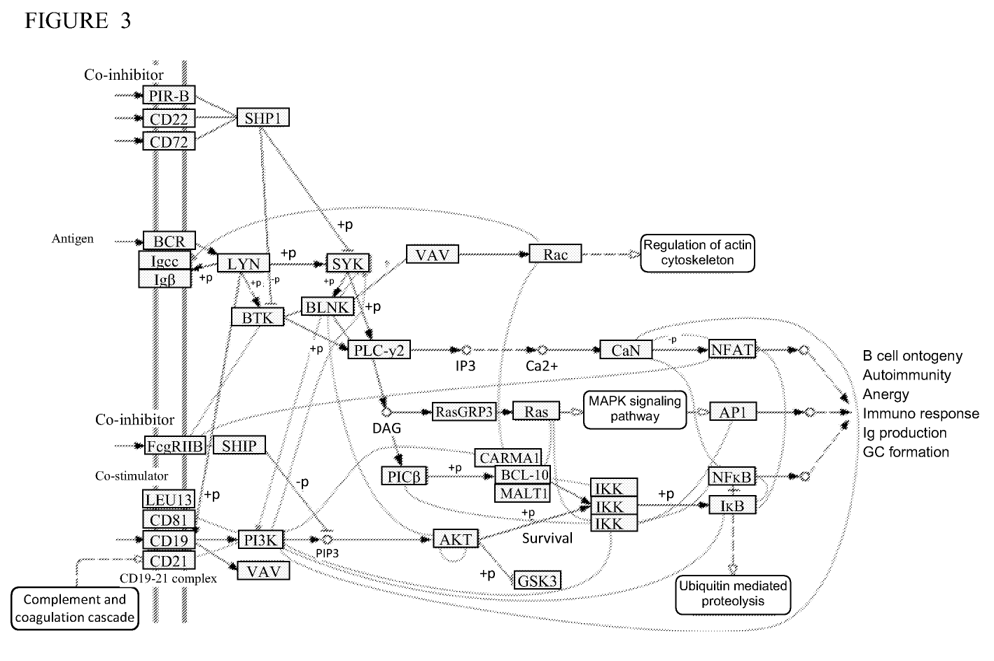

The first group included 4 identical interactions (edges) between nodes. The second group demonstrated 3 interactions with the same topology but an inverted direction. The third group contained 7 interactions with the same neighborhood or Markov blanket. Considering only the first two groups, only 1 out of 7 edges (Gsk3b→Akt2) is annotated as a phosphorylation/dephosphorylation interaction in KEGG and also appears in the consensus network, while the remaining 6 out of 7 edges annotated as transcriptional-level interactions in KEGG appear in the BN. This suggests that the protein modification edges in the KEGG pathway are disproportionately less well conserved in the BN. The top 25 consensus interactions found from 3,281 networks (top 99.99% of marginalized probabilities) were overlain on the KEGG BCR pathway for systematic comparison (FIG. 3).

### Determination of Hidden Genes Involved in BCR Pathway Using the BN+1 Algorithm

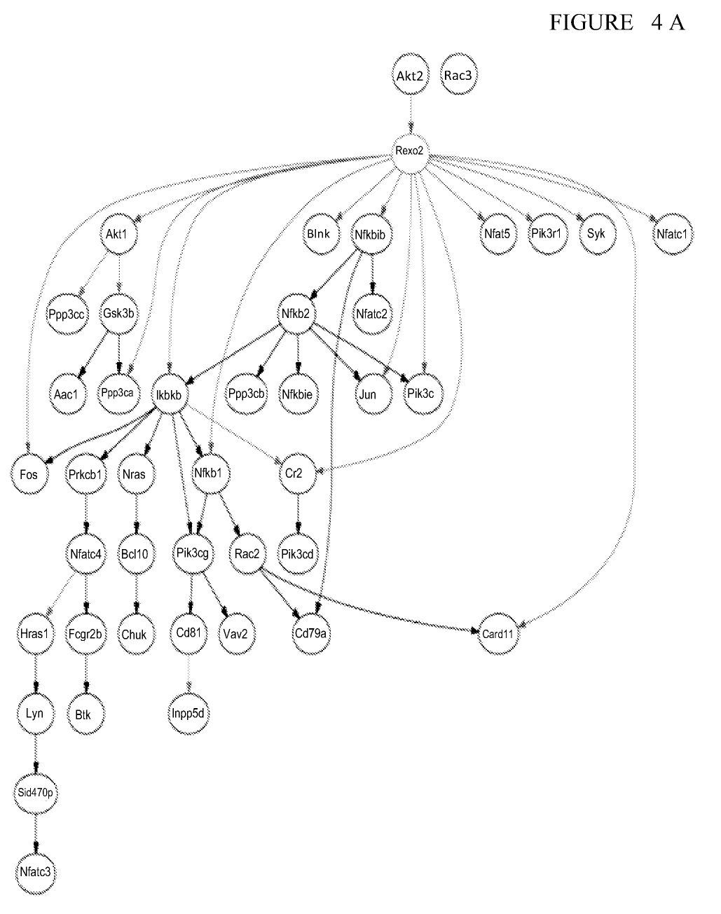

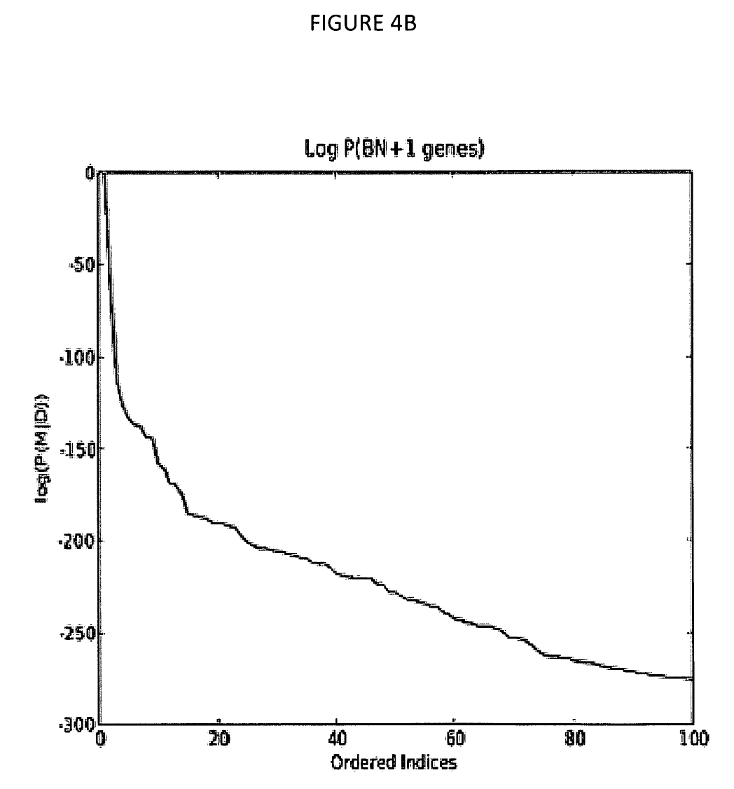

Next, an exemplary analysis was performed wherein the MARIMBA's BN+1 algorithm was used to identify genes that, when added to the KEGG BCR gene set yielded, for example, the highest scoring networks. Many of the genes found on the top-scoring network are associated with BCR. The p-values for the 10 genes indicate they were significant. Surprisingly, the top-scoring BN+1 network containing the gene Rexo2 outperformed the core BN (FIG. 4). In general, adding more variables to a Bayesian network tends to reduce the score of any one network, but not in this case. This top-scoring network suggests that Rexo2 regulates the expression of 14 other genes, covering almost all the key areas of the KEGG BCR pathway.

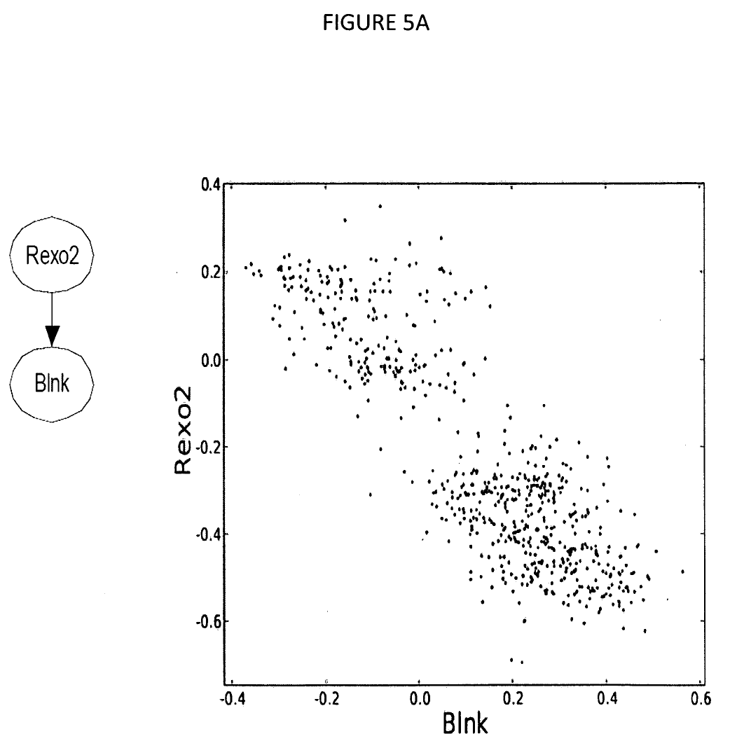

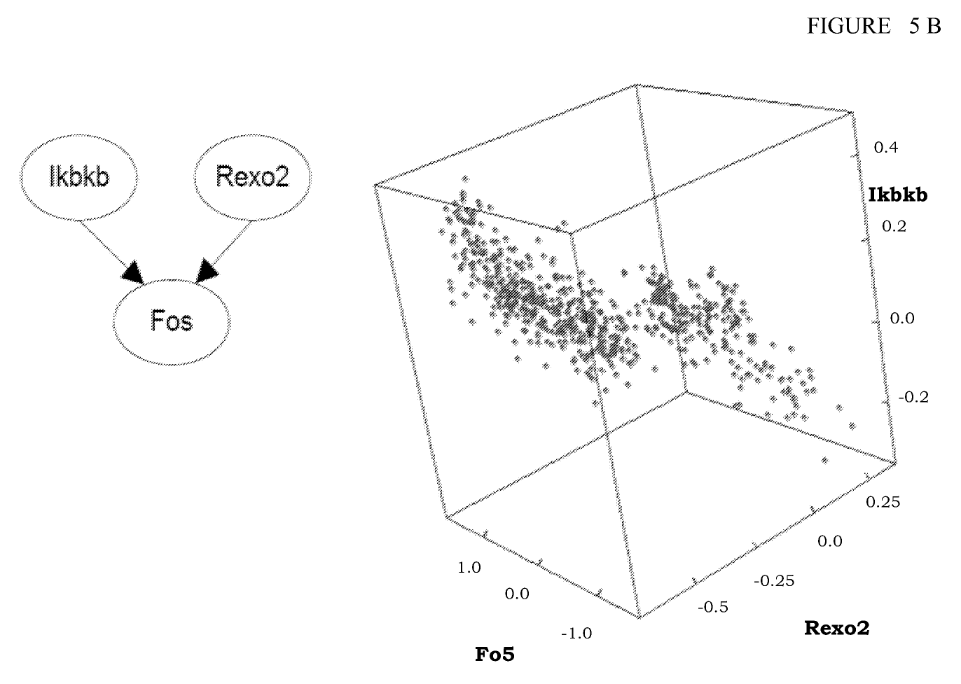

To further explore the role of Rexo2, all Rexo2-related interactions in two or three-dimensional plots of the 625 observations for genes attached to Rexo2 were plotted (FIG. 5). The Rexo2 protein (also called Rex2, RNA exonuclease 2 homolog) is an oligoribonuclease with 3′-to-5′ exoribonuclease activity targeted to small oligoribonucleotides (Ref: toUniprot on ExPasY (http://expasy.org/uniprot/Q9Y3B8). It is likely that Rexo2 influences either mRNA degradation or expression modulation via interfering RNA. If the action of Roxo2 were RNA degradation, we would expect to see an inverse relationship between Rexo2 and the target genes.

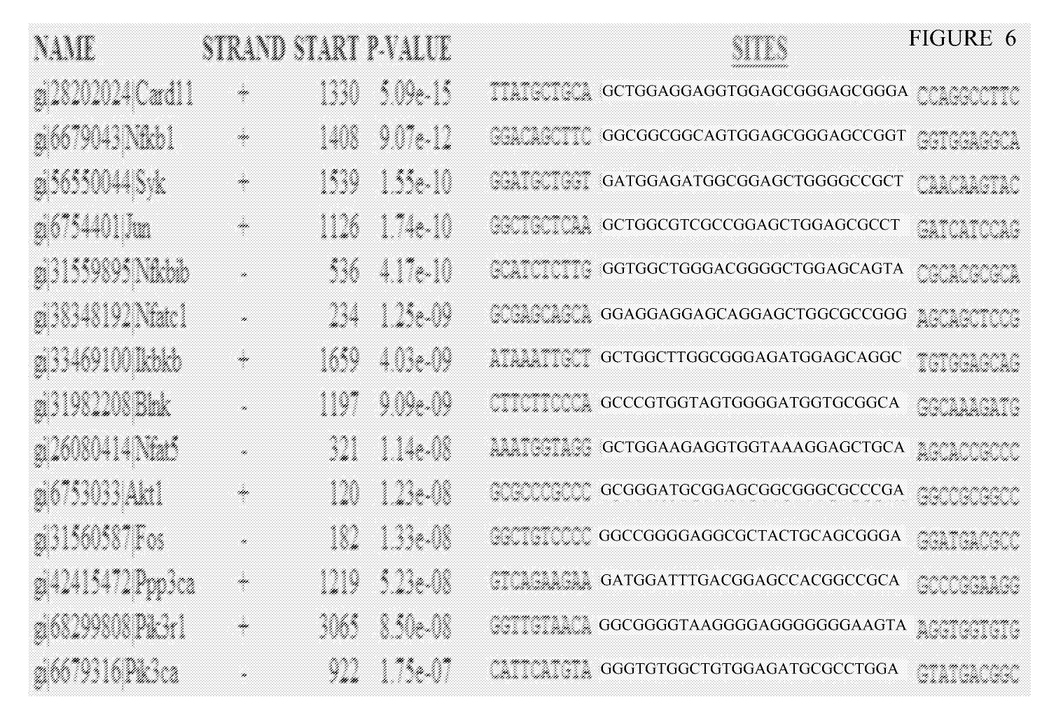

Hierarchical clustering of the forty-three genes by 625-observation BN model containing Rexo2 demonstrated that Rexo2 does not neatly cluster with any of the genes including its targets, suggesting a more complex role for Rexo2. If Roxo2 is acting as a modulator of gene expression via interfering RNA, one would expect to find a region of homology between the target genes that could represent a target for an interfering RNA to bind. Using the online tool MAST (http://meme.sdsc.edu/meme/), a motif was discovered among all the 14 genes directly influenced by Rexo2 (E-value=2.9e-007) (FIG. 6). It was further found that this motif is a conserved microRNA-binding site for a microRNA sequence Mirn718.

The MARIMBA software program was useful in analyzing the BCR pathway using microarray chip data for B cells. The results demonstrated that BN methods trained on microarray data alone identify some but not all transcriptional-level dependencies between genes. For example, the expression profiles derived BN and the KEGG pathways are in agreement about the role of NF-κB. NF-κB is a transcription factor that controls the gene expression of a list of transcribed gene products. The expression data derived BN predicts that Ikbkb (i.e., IKKβ) regulates the expression of several NF-κB related transcripts (including NFκb2, NFκbib, and NFκbie), which agrees with connections observed in the KEGG pathway information (Gloire, 2006). It was also observed that many connections inferred from the expression data are confirmed by literature yet do not appear in the manually curated KEGG pathway database. For example, the BN-inferred interactions between Nfkb2 and IkB members Nfkbib and Nfkbie agree with their literature-documented negative feedback mechanisms (Gloire, 2006). In another example, the BN predicts that Syk, a protein-tyrosine kinase, mediates expression of Nfkbie, an IκB protein—a finding that has strongly been supported by many literature reports (Takada, 2004; Mahabeleshwar, 2003; Takada, 2003) although is absent from the KEGG pathway. These analyses confirm that MARIMBA is able to faithfully detect biologically relevant connections between genes that go beyond simple pathways such as KEGG. However, as demonstrated in Table 1, Bayesian network modeling based on transcriptional gene expression data showed a weak ability to recognize protein-level events such as phosphorylation and dephosphorylation which appear frequently within the KEGG BCR database.

However, these results indicate that the MARIMBA network derived from expression data alone cannot be accurately predicted from the topology of a KEGG pathway. This lack of agreement could be for at least three reasons. First, expression data describes a state of a population of cells, while a pathway describes a more general process. Second, the BCR pathway in KEGG is generally accurate, but may not be relevant in the particular experimental conditions tested here. Third, the KEGG pathway is incomplete, thus some disagreements are attributed to features uncovered from the expression data that arguably should be in the KEGG pathway but have not yet been added.

In developing embodiments of the present invention, the BN+1 algorithm was tested on the AfCS data to determine if any additional genes outside of the BCR KEGG pathway would significantly improve the top BN network model. Analysis of the remaining top 10 BN+1 reveled that many of the detected “hidden” genes are relevant to BCR signaling. The present invention is not limited to a particular mechanism. Indeed, an understanding of the mechanism is not necessary to practice the present invention. Nonetheless, it is contemplated that the hidden genes are acting as cross-talk elements or members of the BCR pathway not in the original KEGG description. The top-scoring gene Rexo2 has not been well studied in the context of the BCR pathway, but it is apparent from the analysis that Rexo2 is a master regulator of the BCR signaling pathway by influencing many genes, at least in the transcriptional level.

It is contemplated that the role of Rexo2 is such that the gene regulates the BCR pathway through an inhibitory RNA-like process. Rexo2 is shown to contain, for example, a substrate binding pocket and a conserved c-terminal endonuclease/exonuclease/phosphatase (EEP) domain (Mian, 2006). It acts as a 3′-to-5′ exoribonuclease specific for small oligoribonucleotides. After searching for a conserved motif across the 14 predicted target genes of Rexo2, a motif was found that closely matches the motif of a mouse microRNA, Mirn718. Mirn718 is a microRNA located on the X chromosome within an exon of the gene encoding interleukin-1 receptor-associated kinase 1 (Irak1, http://microrna.sanger.ac.uk/cgi-bin/sequences/mirna_entry.pl?acc=MI0004707). IRAK1 forms a complex with IL1R1 after induction by IL1 through its association with IL1RAP and subsequently interacting with TRAF6 required for IL-1 mediated NF-κB activation. IRAK-1 is a serine-threonine kinase and acts as the essential upstream adaptor that recruits BCL10 (B cell lymphoma 10) to the TLR4 signaling complex and mediates signaling to NF-κB through the BCL10-MALT1-TRAF6-TAK1 cascade (Dong, 2006). It was also shown that Epstein-Barr virus latent membrane protein 1 (LMP1) activates NF-κB through the IRAK1 pathway, and this activation is critical for Epstein-Barr virus infected B lymphocyte survival (Luftig, 2003). Irak1 in turn is associated with apoptosis and toll-like receptor signaling and is associated with the innate immune response in B-cells.

In some embodiments, the web-based portal of MARIMBA in combination with BN+1 simulations provides a non-computational user the ability to merge existing data from experiments and online pathway databases for use constructing, executing, and analyzing molecular biological pathways. In some embodiments, the integration of microarray expression data and other data types with existing KEGG pathway knowledge maximizes, for example, the reconstructive power of the BN+1 simulations, allowing identification of unknown gene regulatory interactions, removal of spurious inferences, and confirmation of previously-known interactions. The user interface and project/analysis management approach permit large-scale analyses such as BN+1. In some embodiments, the BN+1 algorithm provides for the discovery of biologically relevant hidden factors that connect pathways in a particular cell or tissue type. In some embodiments, the processed AfCS data is stored in the MARIMBA database so that researchers can analyze other pathways using the same datasets using the BN+1 algorithm. In some embodiments, MARIMBA allows direct usage of gene expression data available in the NCBI Gene Expression Omnibus (GEO) system for analysis by the BN+1 algorithm.

In some embodiments, the present invention provides systems and methods for analyzing other kinds of pathway-like data such as protein-protein interaction maps merged with different kinds of experimental data such as proteomic and or metabolomic data. In some embodiments, the present invention provides for analyzing and determining molecular and biochemical pathway information useful in drug target selection. For examples, the BN+1 algorithm (as processed with a software program such as MARIMBA) is used to find new molecular entities that not only better explain the behavior of a pathway, but also can be used as drug targets for clinical therapeutics and research endeavors.

### EXPERIMENTAL

The following examples are provided in order to demonstrate and further illustrate certain preferred embodiments and aspects of the present invention and are not to be construed as limiting the scope thereof.

### Example 1

### MARIMBA System Architecture and Data Flow

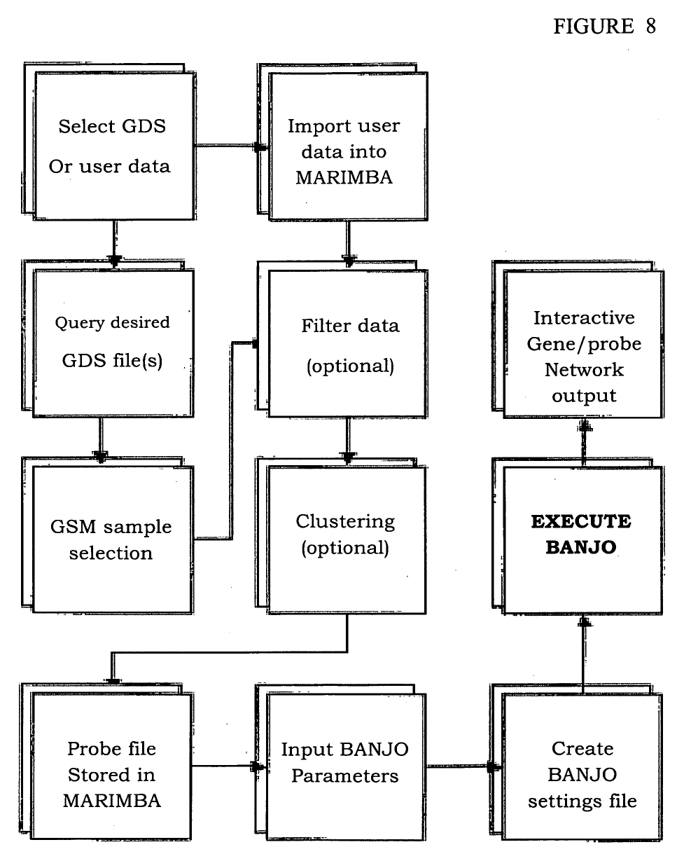

In this example, MARIMBA is implemented using a three-tiered architecture built on two Dell Poweredge 2580 servers that run the Redhat Linux operating system and can be found at http://helab.bioinformatics.med.umich.edu/marimba/index.php. MARIMBA relies on the power of the BANJO (Bayesian Network Inference with Java Objects) system developed by Alex J. Hartemink at Duke University for Bayesian analysis (FIG. 8).

Users submit analysis requests and database queries through the web. These queries are then processed using PHP, Perl, Python, JavaScript, and SQL (middle-tier, application server based on Apache) against a MySQL (version 5.0) relational database (backend, database server). The result of each query is presented to the user in the web browser. An exemplary data pipeline for MARIMBA is shown in FIG. 1. A user can upload microarray/proteomics data or use public NCBI GEO data by simply typing a GEO dataset (GDS) ID. For example, a user enters a gene list for Bayesian network modeling or opts to select genes from a specific KEGG pathway. Data can be preprocessed with available fold change or clustering tools. After selection of modeling parameters, Bayesian networks are searched by simulated annealing or greedy algorithm in BANJO (Smith, 2006) using settings similar to those used in other bioinformatics works (Bose, 2006). Top-scoring networks and consensus networks are displayed graphically in a web-based graphical user interface (GUI) using, for example, software Matplotlib (http://matplotlib.sourceforge.net/) and DOJO (http://dojotoolkit.org/).

To determine if the addition of any single gene improves the top network score when added to the network, the “BN+1” approach is used to recalculate the Bayesian networks by iteratively adding a gene from a defined gene list to an existing top network (prior structure) and subsequent relearning of the network for the new gene list. Each new “BN+1” query per gene is recalculated individually on a compute cluster.

### Example 2

### Processing of AfCS B Cell Microarray Data

The details of the Alliance for Cell Signaling (AfCS) B cell microarray experiment were reported previously (Lee, 2006; Zhu, 2004) and available from the AfCS website. Briefly, B cells purified from splenic preparations from 6- to 8-wk-old male C57BL/6 mice were treated in triplicates or quadruplicates with medium alone, or one or two of 32 different ligands for 0.5, 1, 2, and 4 h (AfCS protocol-PP00000016). RNA was extracted following standard AfCS protocol PP000009. An Agilent cDNA microarray chip that contains 15,494 cDNA probes printed on 15,832 spots was used. It represents 10,615 unique MGI gene matches (Lee, 2006). Each Agilent array was hybridized with Cy5-labeled cDNA prepared from splenic B cell RNA and Cy3-labeled cDNA prepared from RNA of total splenocytes used as an internal reference (AfCS protocol-PP00000019). The arrays were scanned using Agilent Scanner G2505A, and images were processed using the Agilent G2566AA Feature Extraction software version A.6.1.1.

The microarray raw data were downloaded from the AfCS repository at ftp://ftp.afcs.org/pub/datacenter/microarray/marray_bcell_singlelig_agilent_cDNA_txt.ta r.gz. Overall, data from 625 out of 629 unique Agilent microarray chips were selected from double and single-ligand temporal treatment samples as well as control samples. The microarray data were extracted from each of the 625 Agilent chips and combined to create one large data file (625×# probesets on chip). Observations per variable in the BN model were selected as the log ratio of cy5/cy3, which is interpreted as ratio of a given gene in the context of B cells versus the total splenic environment. Hence, each Agilent microarray chip provides one unique observation of relative expression level per selected probe. Though triplicate microarray experiments were available in most cases per unique treatment and time of drug administration, it was assumed that each experiment provides an independent source of information.

### Example 3

### Gene Selection Based on KEGG BCR Pathway

Genes were selected from the KEGG B cell receptor signaling pathway (http://www.genome.jp/dbget-bin/www_bget?path:mmu04662) and converted to Agilent specific probe identities. Forty-two genes were identified using updated AfCS annotation data. KEGG identifiers per gene were acquired using a python SOAP query to the KEGG database. A unique database was constructed in MARIMBA to include AfCS annotations and automate gene selection and gene averaging when requested. KEGG gene symbols are matched in the MARIMBA MySQL database, and converted to respective Agilent probe identifiers. Users opt to either average or select highly-variable probes for representing a respective gene. Probe averaging/selection occur in a final write step. These identifiers were converted to gene symbol using a custom MySQL database loaded with Agilent identifiers.

### Example 4

### Generation and Execution of Bayesian Network (BN) Modeling

Basic MARIMBA-formatted files for use in Bayesian modeling were generated. All 625 individual conditions in the AfCS dataset were selected. Individual conditions can be specified using an interactive webpage. Conditions, included genes, and type of analysis (BN) were selected at this step. This webpage allows verification of all settings, such as method of gene combination (averaging or top probe selection) and type of file write (BN or BN+1 write). During the writing process, BANJO-format data files are generated. In the case of multiple or redundant probeset identifiers per specified gene, averaging was employed. Thus, multiple occurrences were treated as replicates, and were averaged at the respective treatment and time. The final dataset was a BANJO-format data file with 625 rows (unique observations) and 42 columns (42 uniquely-identified genes).

BN simulation settings were selected after completing the data and gene selection processes, respectively. BN files are submitted via the online interface in MARIMBA. Each dataset was transferred to the server prior to transfer to the cluster. On the cluster, a query is based from a controller to one or more agents. The cluster is used to pass new BN and “BN+1” analyses to free agents on the server. Each node in the cluster runs a unique BANJO simulation. The observational file, settings file, and prior knowledge file are passed to the compute nodes. Individual probe lists are retained on the server to protect the integrity and identity of probesets included in an individual analysis. The compute cluster posts status updates to the primary MARIMBA server via a MySQL table. After completion of an individual BN or BN+1 analysis, individual report files are returned to the server.

### Example 5

### Visualization and Interpretation of Bayesian Network Results

Model averaging and equivalence class searching were implemented to determine the “core BN” network model. Here, model averaging was defined as inclusion of an edge between two genes if that edge appeared in more than a specified percentage of the top-scoring networks with identical scores. MARIMBA displays top-scoring networks of BN, BN+1, and combined networks to the user. The BN+1 display environment provides plots for probability of each network in the query, thus enabling comparison of networks for relevance and likelihood.

To allow the user to interpret the relationships predicted by the BN engine, a module is developed to allow a user to generate 2D/3D scatter plots by clicking on a variable node with 1 or 2 other variable nodes as parents. The LiveGraphics3D package (http://www.vis.unistuttgart.de/˜kraus/LiveGraphics3D/) is applied to draw 2D and 3D plots.

### Example 6

### Implementation of “BN+1” Analysis

The core BN network is used as a starting topology in the BN+1 analysis. Probeset selection was designed as above for the standard BN analysis. In addition, probesets not included in the BN structural file were included in the BN+1 list. BN core files, including the BN settings, dataset, probeset list, and report file are used for BN+1 analysis. A unique BANJO analysis is created for each BN+1 probeset. The BN core files are copied from a previous analysis if not present in the current analysis. For the BN+1 analysis, each of the 11 top-scoring core networks from the original BN simulations was used as a starting topology for the BN+1 simulation. Each query took 16.5 minutes per job that allowed an additional sleep time of one minute per query and permits lag time for posting from the grid to server. Network searching was done using simulated annealing. After all jobs successfully post to the server, an additional precompute step was initiated to identify genes which have the best overall top network scores. The total modeling time of all the “BN+1” simulations was greater than 1 month to test all 10,792 matched & unmatched genes (10,344 annotated and 448 genes) using an Apple G5 cluster with 48 nodes.

### Example 7

### Ranking and Probability of Bayesian Network (BN) and BN+1 Results

For BN and BN+1, the probability selecting a given network from the top n uniquely-scoring was calculated, ordered networks using the following marginalization strategy:

$$P ( {Network}_{i} ) = {e}^{Δ {score}_{i}} / {\sum }_{x = 1}^{n} {e}^{Δ {score}_{x}}$$

where the score difference

Δscoreᵢ=Sᵢ−S₁

is the decrease in score between the current log probability score S (S=ln[P (M|D)]) for network i and maximum score S₁ (indices i correspond to ordering by decreasing score value). Log probability scores are produced by BANJO following the algorithm described by Cooper et al. After BN searching concluded, 3550 networks were ranked and ordered via decreasing score. The top 229 non-redundant network scores were identified from these 3550 networks (n=229). In the BN+1 analysis, the first set of BN+1 simulations (out of the 11 possible starting networks) was used to determine the orders. Here, 10,832 best networks were identified and saved, with one best network representing a given annotated or unannotated feature (thus giving 10,832 unique networks, or n=10,832), and later sorted via descending log probability score. Marginalization was executed separately for the BN and BN+1 analyses. Finally, the probabilities for each are shown as semi-log plots.

### Example 8

### Hierarchical Clustering

Clustering tools were selected from Pycluster, a Python interface with a C clustering library (de Hoon, 2004). MARIMBA currently permits k-means and k-median clustering of the working dataset (including probeset and corresponding data file). Graphical results are displayed dynamically for each cluster using the Matplotlib tool (http://matplotlib.sourceforge.net/), including a JPEG image of the individual cluster, checkbox selection of individual clusters, and lists of probesets with links to annotation information. Individual probesets are selected subsequently after cluster selection on a second page.

### Example 8

### Motif Discovery and Search

MEME/MAST server (http://meme.sdsc.edu/meme/meme.html) was used for motif discovery and search among all the 14 genes directly influenced by Rexo2.

### Example 9

### microRNA and Target Search

The miRBase (http://micro ma.sanger.ac.uk/sequences/) was used to search microRNA sequences with possible targets being one or more of the 14 genes directly influenced by Rexo2. A Miranda search was used to find possible targets of microRNAs in murine system.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method for determining new molecular elements in a molecular pathway comprising: applying genes expression data and molecular pathway data to a processor configured to run a BN+1 algorithm under conditions such that said processor uses said BN+1 algorithm to determine new molecular elements in said molecular pathway.

2. The method of claim 1, wherein said gene expression data is microarray gene expression data.

3. The method of claim 1, wherein said molecular pathway is a known molecular pathway.

4. The method of claim 1, wherein said BN+1 algorithm is a component of a computer software program.

5. The method of claim 4, wherein said computer software program is Molecular Annotation Resource for Integrating Microarrays with Bayesian Analysis (MARIMBA) software.

6. The method of claim 1, wherein said determining new molecular elements in said molecular pathway comprises the generation of a Bayesian network from said BN+1 algorithm.

7. The method of claim 1, further comprises displaying molecular element relationships in said molecular pathway as determined by said BN+1 algorithm.

8. The method of claim 7, wherein said displaying comprises dimensional plots.

9. A system comprising a processor configured to receive gene expression data, to receive molecular pathway data, and to run a BN+1 algorithm.

10. The system of claim 9, wherein said system further comprises gene expression data.

11. The system of claim 9, wherein said system further comprises molecular pathway data.

12. The system of claim 9, wherein said system further comprises a processor configured to identify new molecular elements in a molecular pathway using said BN+1 algorithm.

13. The system of claim 9, wherein said processor generates a Bayesian network from said BN+1 algorithm.

14. The system of claim 9, wherein said processor is further configured to display molecular element relationships in said molecular pathway as determined by said BN+1 algorithm.

15. The system of claim 14, wherein said processor displays said molecular element relationships as dimensional plots.


SEQUENCE LISTING (14 sequences)

SEQ ID NO: 1 - DNA, 47 bp, Artificial Sequence
  Synthetic
ttatgctgcagctggaggaggtggagcgggagcgggaccaggccttc

SEQ ID NO: 2 - DNA, 47 bp, Artificial Sequence
  Synthetic
ggacagcttcggcggcggcagtggagcgggagccggtggtggaggca

SEQ ID NO: 3 - DNA, 47 bp, Artificial Sequence
  Synthetic
ggatgctggtgatggagatggcggagctggggccgctcaacaagtac

SEQ ID NO: 4 - DNA, 47 bp, Artificial Sequence
  Synthetic
ggctgctcaagctggcgtcgccggagctggagcgcctgatcatccag

SEQ ID NO: 5 - DNA, 47 bp, Artificial Sequence
  Synthetic
gcatctcttgggtggctgggacggggctggagcagtacgcacgcgca

SEQ ID NO: 6 - DNA, 47 bp, Artificial Sequence
  Synthetic
gcgagcagcaggaggaggagcaggagctggcgccgggagcagctccg

SEQ ID NO: 7 - DNA, 47 bp, Artificial Sequence
  Synthetic
ataaattgctgctggcttggcgggagatggagcaggctgtggagcag

SEQ ID NO: 8 - DNA, 47 bp, Artificial Sequence
  Synthetic
cttcttcccagcccgtggtagtggggatggtgcggcaggcaaagatg

SEQ ID NO: 9 - DNA, 47 bp, Artificial Sequence
  Synthetic
aaatggtagggctggaagaggtggtaaaggagctgcaagcaccgccc

SEQ ID NO: 10 - DNA, 47 bp, Artificial Sequence
  Synthetic
gcgcccgcccgcgggatgcggagcggcgggcgcccgaggccgcggcc

SEQ ID NO: 11 - DNA, 47 bp, Artificial Sequence
  Synthetic
ggctgtccccggccggggaggcgctactgcagcgggaggatgacgcc

SEQ ID NO: 12 - DNA, 47 bp, Artificial Sequence
  Synthetic
gtcagaagaagatggatttgacggagccacggccgcagcccggaagg

SEQ ID NO: 13 - DNA, 47 bp, Artificial Sequence
  Synthetic
ggttgtaacaggcggggtaaggggagggggggaagtaaggtggtgtg

SEQ ID NO: 14 - DNA, 47 bp, Artificial Sequence
  Synthetic
cattcatgtagggtgtggctgtggagatgcgcctggagtatgacggc